SEQUENCE LISTING (6 sequences)

SEQ ID NO: 1 - DNA, 22 bp, Artificial sequence
  primer for Apobec1 gene
tgagtgagtggtggtggtaaag

SEQ ID NO: 2 - DNA, 21 bp, Artificial sequence
  primer for Apobec1 gene
cgaaattcctccagcagtaac

SEQ ID NO: 3 - DNA, 25 bp, Artificial sequence
  primer for Low Density Lipoprotein Receptor gene
accccaagacgtgctcccaggatga

SEQ ID NO: 4 - DNA, 24 bp, Artificial sequence
  primer for Low Density Lipoprotein Receptor gene
cgcagtgctcctcatctgacttgt

SEQ ID NO: 5 - DNA, 25 bp, Artificial sequence
  primer for Low Density Lipoprotein Receptor gene
aggatctcgtcgtgacccatggcga

SEQ ID NO: 6 - DNA, 26 bp, Artificial sequence
  primer for Low Density Lipoprotein Receptor gene
gagcggcgataccgtaaagcacgagg

(19) United States
(12) Patent Application Publication
(10) Pub. No.: US 2011/0217293 A1
(43) Pub. Date: Sep. 8, 2011

(54) Uses of morelloflavone

(76) Inventors: Kenichi Fujise, Galveston, TX (US); Nongporn Towatana, Hat Yai (TH); Wilawan Mahabusarakam, Songkhia (TH)

(21) Appl. No.: 12/931,310

(22) Filed: Jan. 28, 2011

Related U.S. Application Data
(63) Continuation-in-part of application No. PCT/US2009/000436, filed on Jul. 28, 2009.
(60) Provisional application No. 61/137,256, filed on Jul. 29, 2008.

(51) Int. Cl. A61K 39/395; A61K 31/352; A61K 31/40; A61K 31/4418; A61K 31/405; A61K 31/47; A61K 31/505; A61K 31/616; A61K 31/4365; A61K 31/519; A61P 9/10; A61P 3/06; A61P 7/02

(52) U.S. Cl. 424/133.1; 514/456; 514/423; 514/277; 514/415; 514/311; 514/275; 514/161; 514/301; 514/262.1

(57) ABSTRACT

Provided herein are methods of treating postangiplasty, in-stent restenosis or atherosclerosis in an individual in need of such treatment, comprising the step of administering to said individual an effective dose of morelloflavone with or with a effective dose of one or more of an HMG-CoA reductase inhibitor or a hypolipidemic agent or lipid-lowering agent or other lipid agent or lipid modulating agent or anti-atherosclerotic agent. Also provided are pharmaceutical compositions comprising a morelloflavone or pharmaceutical combinations comprising a morelloflavone and one of an HMG-CoA reductase inhibitor or a hypolipidemic agent or lipid-lowering agent or other lipid agent or lipid modulating agent or anti-atherosclerotic agent.

### CROSS-REFERENCE TO RELATED APPLICATIONS

This is a continuation-in-part application which claims benefit of priority under 35 U.S.C. §120 of pending international application PCT/US2009/004346, filed Jul. 28, 2009, which claims benefit of priority under 35 U.S.C. §119(e) of provisional application U.S. Ser. No. 61/137,256, filed Jul. 29, 2008, now abandoned, the entirety of both of which are hereby incorporated by reference.

### FEDERAL FUNDING LEGEND

This invention was produced in part using funds obtained through grant NIH HL04015 and HL68024. Consequently, the federal government has certain rights in this invention.

### BACKGROUND OF THE INVENTION

1. Field of the Invention

The present invention relates generally to the fields of medicine and cell biology. More specifically, the present invention relates to oral anti-restenotic agent and uses thereof.

2. Description of the Related Art

A major disadvantage of percutaneous coronary intervention (PCI) is restenosis or renarrowing of dilated or stented arteries—caused primarily by the migration and proliferation of b-actin-immunoreactive vascular smooth muscle cells (1-3).

The most commonly used preventive drug treatment for atherosclerosis is HMG-CoA reductase inhibitor (statins). However, 50% of patients that come to the ER with acute heart attacks have normal cholesterol levels. Thus, statins by themselves would not eliminate either atherosclerosis or complications of atherosclerosis, such as heart attack, stroke, aneurysm, peripheral artery diseases.

The most commonly used preventive method for atherosclerosis is drug-eluting stents. Although highly effective, patients with drug-eluting stents must take clopidogrel at least for 12 months, if not longer. The safety of drug-eluting stents without clopidogrel therapy has not been fully established. Because of this safety concern, many patients wish to have bare-metal stents. Bare-metal stents is associated with higher rates of restenosis but patients can stop taking clopidogrel after 3 months.

Garcinia dulcis (FIG. 1A), a plant that belongs to the Guttiferae family, is widely distributed in Thailand, and other Southeast Asian countries (4). Also known as maphuut in Thailand and mundu in Indonesia and Malaysia, G. dulcis has been used in traditional medicine for centuries (5). While several bioactive compounds have been isolated from the plant (5-6), the main constituent of the leaves of G. dulcis is morelloflavone (5, 7, 4′, 5″, 7″, 3″, 4″-heptahydroxy-[3,8″]-flavonylflavanone, CAS Registry No. 16851-21-1), a biflavonoid comprising two covalently linked flavones, apigenin and luteolin (7) (FIG. 1B). Despite the extensive medicinal use of G. dulcis, the biological activities of morelloflavone have not been evaluated in detail with only a few published studies (8-11).

There is a need in the art for improved oral anti-restenotic and antiatherosclerotic pharmacologic therapies. The present invention fulfills this long-standing need and desire in the art.

### SUMMARY OF THE INVENTION

The present invention is directed to a method for treating postangiplasty or in-stent restenosis in an individual in need of such treatment. The method comprises administering to said individual an effective dose of morelloflavone. The present invention is directed to a related invention comprising a further method step of administering an HMG-CoA reductase inhibitor.

The present invention is directed to a related method for treating atherosclerosis in an individual in need of such treatment. The method comprises administering to said individual an effective dose of morelloflavone. The present invention is directed to a related invention comprising a further method step of administering an HMG-CoA reductase inhibitor.

The present invention also is directed to a method for treating postangiplasty or in-stent restenosis in an individual in need of such treatment. The method comprises administering to said individual an effective dose of morelloflavone in a dose of from about 0.1 mg/kg to about 100 mg/kg of the individual's body weight. The present invention is directed to a related invention comprising a further method step of administering a platelet aggregation inhibitor, an HMG CoA reductase inhibitor or a hypolipidemic agent or lipid-lowering agent or other lipid agent or lipid modulating agent or anti-atherosclerotic agent.

The present invention is directed further to a method for treating postangiplasty or in-stent restenosis in an individual in need of such treatment. The method comprises administering to said individual an effective dose of morelloflavone and an HMG-CoA reductase inhibitor. The present invention is directed to a related invention comprising a further method step of administering a platelet aggregation inhibitor.

The present invention is directed further still to a pharmaceutical composition comprising a morelloflavone and a pharmaceutically acceptable carrier therefore.

The present invention is directed further still to a pharmaceutical composition comprising a morelloflavone and one of a HMG CoA reductase inhibitor compound, a hypolipidemic agent or lipid-lowering agent or other lipid agent or lipid modulating agent or anti-atherosclerotic agent, or a platelet aggregation inhibitor.

Other and further objects, features, and advantages will be apparent from the following description of the presently preferred embodiments of the invention, which are given for the purpose of disclosure.

### BRIEF DESCRIPTIONS OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIGS. 1A-1F show that morelloflavone does not affect cell cycle progression and causes no cytotoxicity or apoptosis in vascular smooth muscle cells. FIG. 1A shows Garcinia dulis. L, the leaves; F, fruit; FL, flower. FIG. 1B shows the structure of morelloflavone. Morelloflavone (MW=556), a biflavonoid, consists of two flavones covalently linked to each other. FIG. 1C shows the purification and characterization of morelloflavone. The current preparation of morelloflavone was purified from the leaves of Garcinia dulis and found to be 93.4% pure as determined by HPLC. FIG. 1D shows a flow cytometric analysis. Cell cycle progression was evaluated by treating vascular smooth muscle cells with various concentrations (0-100 mM) of morelloflavone and subjecting them to flow cytometric analyses (representative data from 3 independent experiments). FIG. 1E shows a BrdU assays. The percentage of S-phase cells were determined by treating vascular smooth muscle cells with various concentrations (0-100 mM) of morelloflavone and measuring the uptake of BrdU by these cells (n=4). Morelloflavone does not affect the percentage of S-phase cells at a concentration equal to or less than 10 mM (P=0.079 by one-way ANOVA on BrdU labelling indices between 0-10 mM morelloflavone). Morelloflavone, at 100 mM, decreases BrdU labelling indices (****, P<0.001 for BrdU labelling indices between cells treated with 10 and 100 mM morelloflavone). FIG. 1F shows a MTT (3-[4,5-Dimethylthiazol-2-yl]-2,5-diphenyltetrazolium bromide) vascular smooth muscle cell viability assay. Morelloflavone is not cytotoxic to vascular smooth muscle cells at concentrations up to 10 mM (NS [P=0.071] for MTT survival rate [%] among 0, 0.01, 0.1, 1 and 10 mM, by one-way ANOVA; ****, P<0.001 for MTT survival rate (%) between 10 and 100 mM morelloflavone; n=4). FIG. 1G shows a DNA fragmentation assay. DNA fragmentation indices decreased as morelloflavone concentrations increased (n=2 each for 0, 1, 10, and 100 mM; *, P=0.032, by one-way ANOVA). Morelloflavone does not cause apoptosis at concentrations equal to or less than 100 mM.

FIGS. 2A-2F show that morelloflavone inhibits vascular smooth muscle cell migration, invasion and lamellipodium formation. In a scratch wound cell migration assay, FIG. 2A shows photomicrographs of migration patterns of morelloflavone-treated vascular smooth muscle cells and FIG. 2B shows migration indices. Migration indices were calculated as migrated cells per unit area (cells/mm²). P<0.001 by one-way ANOVA (n=5). In a modified Boyden chamber invasion assay FIG. 2C shows photomicrographs of invasion patterns of vascular smooth muscle cell in the presence and absence of morelloflavone (1 mM) and FIG. 2D shows the effect of morelloflavone on vascular smooth muscle cells migration. Migrated cell numbers represented the total cell numbers on each test sites (8.0 [mm²]). Morelloflavone at 1 mM significantly blocked vascular smooth muscle cell migration (P=0.002 by two-way ANOVA, n=5). SMGS, smooth muscle cell growth supplement (Cascade Biologics, Portland, Oreg.). In a Lamellipodia formation assay, FIG. 2E shows confocal microscopy of vascular smooth muscle cells stimulated by sera in the presence of various concentrations of morelloflavone (0-10 mM). Arrow, lamellipodia; size bar, 50 mm and FIG. 2F shows Lamellipodium indices calculated as the number of lamellipodia divided by the total number of cells counted. Open bar, no serum; closed bar, 5% serum. Serum stimulation significantly increased lamellipodium indices (P<0.005, n=3) in the absence of morelloflavone. Serum stimulation failed to increase lamellipodium indices in the presence of 1-10 mM morelloflavone. Morelloflavone significantly decreased lamellipodium indices in a dose-dependent fashion (P<0.001 by one-way ANOVA).

FIGS. 3A-3C show that morelloflavone inhibits multiple migration-related kinases. FIG. 3A shows the phosphorylation of FAK and c-Src. FAK, total focal adhesion kinase; p-FAK, phosphorylated FAK; c-Src, total c-Src; p-c-Src, phosphorylated c-Src. Morelloflavone robustly decreased phosphorylation of FAK and c-Src. The posphorylation indices of a certain kinase (such as p-FAK/FAK) were calculated by dividing the signal intensity of the band of the phosphorylated kinase by the signal intensity of the band of the total kinase, at a given morelloflavone concentration. The indices of untreated cells were normalized to 100. FIG. 3B shows the phosphorylation of ERK. ERK, total ERK; p-ERK, phosphorylated ERK; Morelloflavone robustly decreased phosphorylation of ERK. FIG. 3C shows the activation of RhoA, Rac1, and Cdc42. Morelloflavone blocks the activation of RhoA at 0.1 mM, Cdc42 at 10 mM; but it has no significant effect on Rac1 or Cdc42 activation.

FIGS. 4A-4D show that morelloflavone inhibits injury-induced neointimal proliferation in a mouse carotid artery injury model. FIG. 4A are photomicrograms of the carotid arteries showing Verhoeff-van Gieson (VVG) staining of mouse carotid arteries. Uninjured, right carotid arteries that are sham operated; injured, left carotid arteries in which endothelial cells were denuded by the insertion of an epoxy resin probe; Arrows, neointimal formation. FIG. 4B shows morphometric analyses of injured and uninjured mouse carotid arteries. P<0.01 (Two-sample t-test; n=9 for control; n=10 for morelloflavonetreated). Morelloflavone significantly blocked injury-induced neointimal formation in mouse carotid arteries. FIGS. 4C-4D show TUNEL apoptosis assays. The TUNEL indices were determined as the number of cells with TUNEL-positive nuclei divided by the total number of cells counted and expressed as a percentage (n=9 for control; n=10 for morelloflavone-treated). Size bar=25 mm. FIGS. 4E-4F show Ki-67 proliferation assays. The Ki-67 indices were determined as the number of cells with Ki-67-positive nuclei divided by the total number of cells counted and expressed as a percentage. Size bar=25 mm. Oral morelloflavone treatment resulted in reduced neointimal formation, without increasing apoptotic or proliferating cells in the neointima. FIG. 4G show p-ERK staining. Phosphorylated ERK was detected by using anti-p-ERK antibody. Size bar=25 mm. The nuclear p-ERK signals (black arrows) were seen in 42.9% of the neointima of control animals and in 12.5% of the neointima of morelloflavone-treated animals (n=7 and 8, respectively). Oral morelloflavone treatment was associated with reduced p-ERK positivity in the neointima.

FIGS. 5A-5B show that morelloflavone reduces atherosclerosis in a mouse model of atherosclerosis—En face analyses. FIG. 5A shows atherosclerotic lesions in aortic of Ldlr^{-/-}Apobec1^{-/-} mice. Mice were fed a normal rodent diet or 0.003% morelloflavone-containing diet with for 8 months. Gross view of aortas stained with Oil Red O; red indicates the presence of atherosclerotic lesions. FIG. 5B the degree of atherosclerosis by en face method. The total area affected by atherosclerosis is substantially lower in morelloflavone-treated mice than in control mice (N=12), P=0.0025.

FIGS. 6A-6B show that morelloflavone reduces atherosclerosis in a mouse model of atherosclerosis—cross-sectional analyses. FIG. 6A is a cross-sectional aortic atherosclerosis in Ldlr^{-/-}Apobec1^{-/-} mice. Mice were fed a normal chow diet or morelloflavone—containing diet for 8 months. Representative HE-stained sections from the aortic valve area of Ldlr^{-/-}Apobec1^{-/-} mice fed with normal diet or morelloflavone—containing diet (N=6). FIG. 6B shows atherosclerotic lesion area by cross-sectioned methods. Quantitative estimation of aortic atherosclerotic lesion involvement. Each data point represents total area of atherosclerotic lesion involvement in aortic valve area (N=6).

FIGS. 7A-7C show that morelloflavone reduces the infiltration of smooth muscle cells into atherosclerotic tissue. The sections (N=7) were stained for SMA and couterstained with hematoxylin (FIG. 7A). Quantitative analysis of SMA-positive cells per section (FIG. 7B) and the SMA index (FIG. 7C) in atherosclerotic lesion is shown.

FIGS. 8A-8C show that morelloflavone does not change the infiltration of macrophages into atherosclerotic tissue. The sections (N=7) were stained for F4/80 and couterstained with hematoxylin (FIG. 8A). Quantitative analysis of F4/80-positive cells per section (FIG. 8B) and the F4/80 index (FIG. 8C) in atherosclerotic lesion are shown.

FIGS. 9A-9C show that morelloflavone does not affect the cell proliferation of the cells within atherosclerotic tissue. The sections (N=7) were stained for Ki67 and couterstained with hematoxylin (FIG. 9A). Quantitative analysis of Ki67-positive cells per section (FIG. 9B) and the Ki-67 index (FIG. 9C) in atherosclerotic lesion are shown.

FIGS. 10A-10B show that morelloflavone does not affect the apoptosis of the cells within atherosclerotic tissue. The sections (N=7) were stained for TUNEL staining and couterstained with hematoxylin (FIG. 10A). Quantitative analysis of TUNEL-positive cells per section (FIG. 10B) and the TUNEL index (FIG. 10C) in atherosclerotic lesion are shown.

### DETAILED DESCRIPTION OF THE INVENTION

As used herein, the term, “a” or “an” may mean one or more. As used herein in the claim(s), when used in conjunction with the word “comprising”, the words “a” or “an” may mean one or more than one. As used herein “another” may mean at least a second or more. As used herein, the term “subject” refers to any target of the treatment. These methods may be used to treat any subject, preferably a mammal, more preferably a human.

In one embodiment of the present invention there is provided a method for treating postangiplasty or in-stent restenosis in an individual in need of such treatment, comprising the step of administering to the individual an effective dose of morelloflavone. Generally, the morelloflavone retards the progression of atherosclerosis, inhibits the migration of vascular smooth muscle cells, and decreases activation of ERK, RhoA, Rac, FAK and cSrc. Generally, an individual who will benefit from such treatment is an individual at risk for percutaneous coronary intervention, i.e., who has undergone a percutaneous coronary intervention or will undergo a percutaneous coronary intervention. A representative individual to be treated has restenosis and/or has atherosclerosis in coronary arteries, cerebral arteries, renal arteries, aorta, or peripheral arteries.

A benefit of morelloflavone is that it may be administered orally. Typically, the morelloflavone is administered is a dose of from about 0.1 mg/kg to about 100 mg/kg of the individual's body weight but a person having ordinary skill in this art might find it advantageous to increase or decrease the concentration of morelloflavone. This method may further comprise the step of administering an HMG-CoA reductase inhibitor or statin.

Representative statins include but are not limited to Atorvastatin, Cerivastatin, Fluvastatin, Lovastatin, Mevastatin, Pitavastatin, Pravastatin, Rosuvastatin, Simvastatin, Simvastatin.

In another embodiment of the present invention there is provided a method for treating postangiplasty or in-stent restenosis in an individual in need of such treatment, comprising the step of administering to the individual an effective dose of morelloflavone in a dose of from about 0.1 mg/kg to about 100 mg/kg of the individual's body weight.

Further to this embodiment the present invention provides a method for treating postangiplasty or in-stent restenosis in an individual in need of such treatment, comprising the step of administering to the individual an effective dose of morelloflavone and an HMG-CoA reductase inhibitor or a hypolipidemic agent or lipid-lowering agent or other lipid agent or lipid modulating agent or anti-atherosclerotic agent. Representative hypolipidemic agents or lipid-lowering agents or other lipid agents or lipid modulating agents or anti-atherosclerotic agents include but are not limited to 1,2,3 or more MTP inhibitors, squalene synthetase inhibitors, fibric acid derivatives, PPAR .alpha. agonists, PPAR dual .alpha./.gamma. agonists, PPAR .delta. agonists, ACAT inhibitors, lipoxygenase inhibitors, cholesterol absorption inhibitors, ileal Na.sup.+/bile acid cotransporter inhibitors, upregulators of LDL receptor activity, cholesteryl ester transfer protein inhibitors, bile acid sequestrants, or nicotinic acid and derivatives thereof, ATP citrate lyase inhibitors, phytoestrogen compounds, an HDL upregulators, LDL catabolism promoters, antioxidants, PLA-2 inhibitors, antihomocysteine agents, HMG-CoA synthase inhibitors, lanosterol demethylase inhibitors, or sterol regulating element binding protein-I agents. The method of the present invention may further comprise administering a platelet aggregation inhibitor. Representative platelet aggregation inhibitors include but are not limited to aspirin, clopidogrel, ticlopidine, dipyridamole, ifetroban, abciximab, tirofiban, eptifibatide, anagrelide, CS-737, melagatran, ximelagatran, and razaxaban.

In yet another embodiment of the present invention there is provided a pharmaceutical composition comprising a morelloflavone and a pharmaceutically acceptable carrier therefor. The present invention is also directed to a pharmaceutical combination comprising a morelloflavone and a HMG CoA reductase inhibitor compound. The present invention is also directed to a pharmaceutical combination comprising a morelloflavone and a hypolipidemic agent or lipid-lowering agent or other lipid agent or lipid modulating agent or anti-atherosclerotic agent. The present invention is further directed to a pharmaceutical combination comprising a morelloflavone and a platelet aggregation inhibitor.

The present invention demonstrates that (1) morelloflavone does not affect cell cycle progression, both by FACS and BrdU assays; (2) morelloflavone does not induce cytotoxicity, both by MTT and DNA fragmentation assays; (3) morelloflavone blocks vascular smooth muscle cells migration, invasion, and lamellipodia formation. Morelloflavone also does not affect cell attachment; (4) morelloflavone blocks FAK, Src, ERK, and RhoA without affecting Rac1 or Cdc42; (5) oral administration of morelloflavone blocks restenosis in mice, without inducing cell cycle arrest or apoptosis. Serum concentrations of morelloflavone were 1.4 mM as measured by HPLC. Furthermore, oral morelloflavone was associated with less ERK activation in the neointima.

The following examples are given for the purpose of illustrating various embodiments of the invention and are not meant to limit the present invention in any fashion.

### Example 1

### Preparation of Morelloflavone

The purification of morelloflavone was performed as described (1) with following modifications. Dried G. dulcis leaves were finely powdered and extracted with acetone. Insoluble matter was removed by filtration, and the filtrate was concentrated in vacuo. A second extraction was achieved with hexane, and the hexane-insoluble fraction was subsequently extracted with dichloromethane. The greenish-yellow residue from the dichloromethane-insoluble fraction was subjected to quick-column chromatography on silica 60H and eluted with dichloromethane-acetone in a polarity gradient manner.

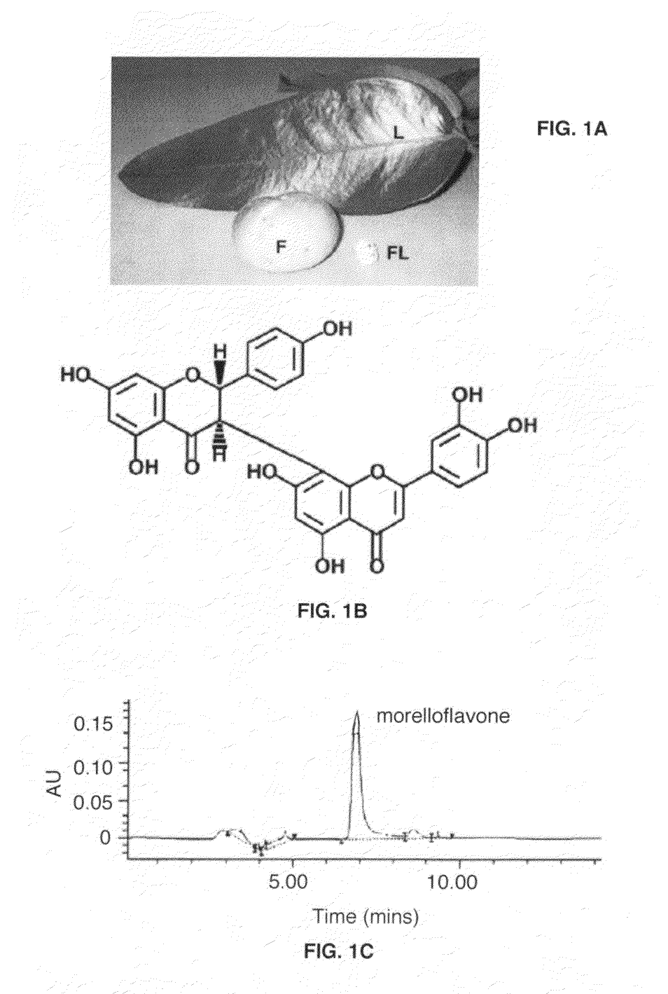

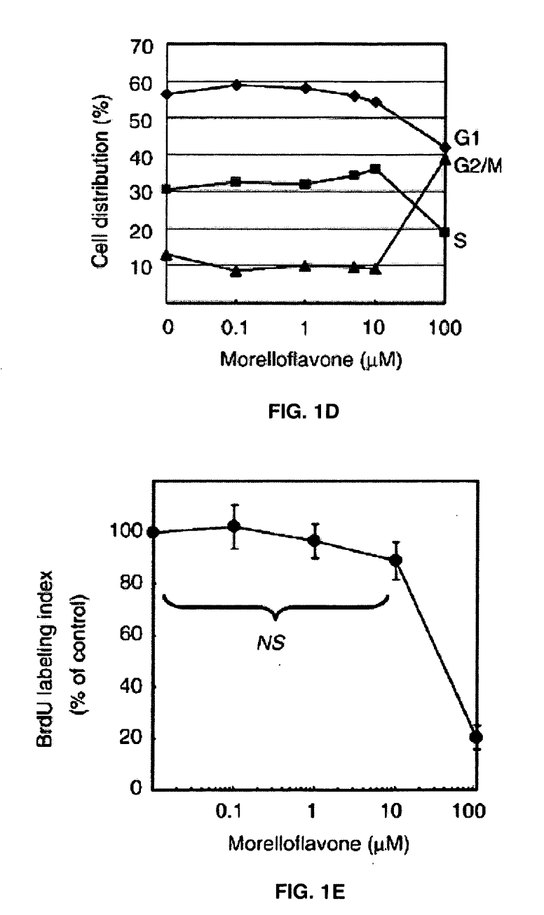

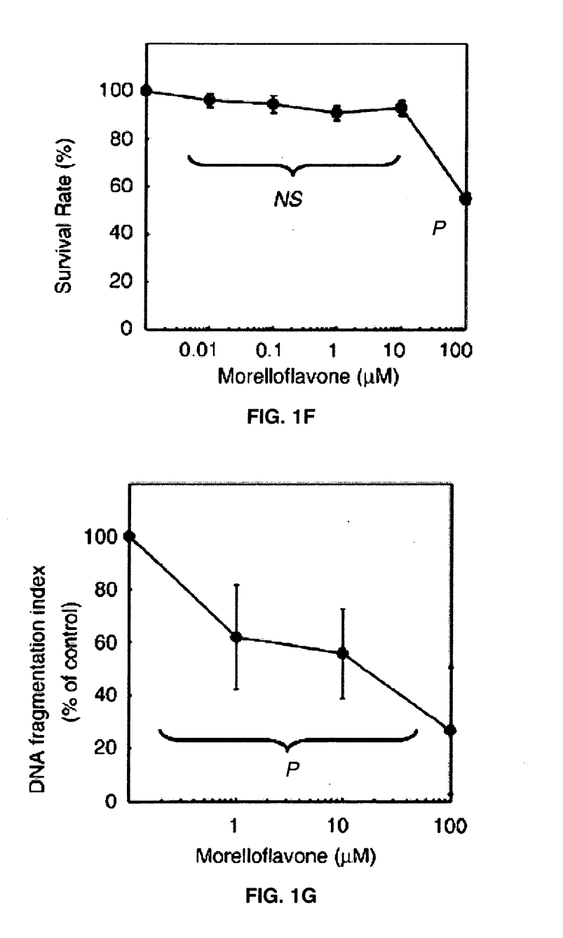

The eluted fractions were combined on the basis of thin-layer chromatography (TLC) results. Finally, the purified compound was concentrated in vacuo, dried, and ground. TLC was used to confirm the desirable fraction for every step of extraction and purification. The purity of this compound was determined by using an HPLC system (Agilent 1100 Series, Germany), equipped with a solvent delivery pump (BinPump G1312A), an autosampler (ALS G1313A), a photodiode-array detector (DAD G1315B) and data output (LC Chemstation, Rev. A.10.02). An ODS-2 column (5 mm particle size, 4.6×2 50 mm i.d.; Inertsil™, Shimadzu, Japan) was used. The mobile phase, consisting of 45% (v/v) acetonitrile and 55% (v/v) of 1% acetic acid, was pumped at a flow rate of 1 ml/min, and the effluent was monitored at 289 nm. Morelloflavone in the sample was identified by comparing its spectral data with that of a standard that had been previously purified from G. dulcis flowers (2). The peak analysis also revealed that the sample contained mostly morelloflavone (94.3%). The purity of the preparation was determined to be 94.3% by using an HPLC system (FIG. 1C) (12).

### Example 2

### Cell Culture

Mouse vascular smooth muscle cells, isolated as described (13), were maintained in 231 media (Cascade Biologics, Portland, Oreg.) supplemented with SMGS (Cascade Biologics) in a humidified incubator at 37° C. with 5% CO₂. Cells from passages 4-9 were used in all experiments. All experiments were performed in subconfluent, unsynchronized cells growing in SMGS except for the lamellipodium formation assay.

### Example 3

### Cell Cycle Analyses

Vascular smooth muscle cells (1×106) were seeded onto 10-cm dishes and treated with various concentrations of morelloflavone. After 24 hr incubation, the cells were fixed with 70% ethanol at 4° C. overnight, treated with RNAse in PBS, stained with propidium iodide (Sigma, St. Louis, Mo.), and subjected to flow cytometric DNA content analysis using Epics XL (Beckman-Coulter, Miami, Fla.). The percentages of cells in G1, S, and G2/M phases were determined using Multi-cycle system software (Phoenix Flow System, San Diego, Calif.).

### Example 4

### BrdU Incorporation Assay

Vascular smooth muscle cells were seeded at 2×10⁴ cells per well in 96-well culture plates and incubated overnight. The next day, cells were found approximately 60-70% confluent. Cells were treated with various concentrations of morelloflavone (0 to 100 mM) for 2 hrs and then pulsed with BrdU at 1 mM for 8 hrs. The amount of BrdU incorporated into the cells was quantified by BrdU Cell Proliferation Assay kit (Calbiochem, San Diego, Calif.), according to manufacturer's instructions. Briefly, the cells were fixed and permeabilized on tissue culture plastic, and incubated with anti-BrdU monoclonal antibody. After extensive washing, bound antibodies were visualized by goat anti-mouse IgG conjugated to horseradish peroxidase and tetra-methylbenzidine substrate and quantified by spectrophotometer at 450 nm. The protein content was determined using the Bradford assay (Bio-Rad, Hercules, Calif.).

### Example 5

### MTT Cell Death Assay

Vascular smooth muscle cells were plated at 1×10⁴ cells per well in 96-well culture plates and incubated overnight. The cells were treated with various concentrations of morelloflavone for 48 hrs. Then, the cells were exposed to MTT labeling reagent at 10 mg/mL for 4 hrs and solubilized in 0.01 N HCl containing 10% SDS overnight. Formed formazan was measured via spectrophotometry at 600 nm.

### Example 6

### DNA Fragmentation Assay

Vascular smooth muscle cells were seeded at 1×10⁵ cells per well in 24-well culture plates, treated with morelloflavone, and subjected to DNA fragmentation assay, according to the manufacturer's instructions (Cell Death Detection ELISAPLUS, Roche). Briefly, the cells were treated with 0-100 mM of morelloflavone for 24 hrs. The 1×10⁵ cells were lysed, cleared by centrifugation, and transferred into streptavidin-coated plates. Anti-histone antibody conjugated to biotin and anti-nucleosomal-DNA-antibody conjugated to horseradish peroxidase were added. After incubation and washing, 2,2′-azino-bis-[3-ethylbenzthiazoline-6-sulfonic acid] (ABTS) substrate solution was added. The 5 absorbance rate at 405 nm against ABTS solution was measured with the reference wavelength of 490 nm.

### Example 7

### Scratch Wound Assay

Vascular smooth muscle cells that had been grown to confluence in 6-well culture plates were scratched with a sterile 1000 ml pipette tip and exposed to various concentrations of morelloflavone. The cells were allowed to migrate onto the plastic surface for 18 hrs and photographed. A migration index was determined based on the number of cells that migrated onto 1 mm² free plastic surface, using the Image J software (NIH).

### Example 8

### Invasion Assay

The lower chambers of the ChemoTx® Disposable Chemotaxis System (NeuroProbe, Gaithersburg, Md.) were filled with 29 ml of SMGS diluted in 231 medium at the appropriate concentrations (0-10%). A filter membrane (5 mm pore size) was positioned over the lower wells, and 1×104 vascular smooth muscle cells suspended in 20 mL 231 medium (without morelloflavone) were placed on the test sites within circular hydrophobic masks on the upper side of the filter plate (n=5 each). Cells were allowed to attach the porous membrane surface for 4 hr when solution covering cells was exchanged to 231 medium either containing 1 mM of morelloflavone or vehicle (0.1% DMSO). After additional 8-hour incubation, cells on the upper surface of the membrane, i.e., cells that had not migrated, were scraped off by Q-tips, and cells that had migrated to the lower surface were fixed and stained by hematoxylin solution and counted. Migrated cell 6 numbers were calculated as the number of cells migrated per 8.0 mm² test site surface area.

### Example 9

### Lamellipodium Formation Assay

The lamellipodium formation assay was performed as described (14). In brief, mouse vascular smooth muscle cells (serum-starved for 24 hrs) were seeded onto fibronectin-coated wells of chamber slides (CultureSlide, BD BioCoat Fibronectin) and incubated with various concentrations of morelloflavone (0, 1, and 10 mM) in the presence or absence of serum for 3 hrs at 37° C. Then, cells were fixed with 4% paraformaldehyde, permeabilized with 0.1% Triton X-100, stained with Alexa Fluor 594-Phallidin (Invitrogen-Molecular Probes) and DRAQ5 (Biostatus, Ltd, Leicestershire, UK), and viewed with the use of a Leica DM6000 confocal microscope. Lamellipodium indices were calculated as the number of lamellipodia divided by the total number of cells counted.

### Example 10

### Western Blot Analysis

For the evaluation of phosphorylated FAK and c-Src, Western blot analyses were performed as described (15). Briefly, vascular smooth muscle cells were seeded on 10-cm dishes, treated with various concentrations of morelloflavone for 5 min, and harvested into RIPA buffer with protease inhibitor cocktails. Cleared cell lysates (500 mg of protein) were incubated with anti-FAK or anti-c-Src antibodies at 4° C. for 1 hr in a final volume of 1 mL RIPA buffer, and incubated for another 1 hr with protein A/G agarose beads (Santa Cruz ; SCBT sc-2003). Immunoprecipitated proteins were eluted into SDS-loading 7 buffer and subjected to 12% SDS-PAGE and immunoblotting using anti-FAK (Santa Cruz; A-17; sc-557) and anti-c-Src (Santa Cruz; SRC-2; sc-18) antibodies for total FAK and c-Src, respectively, and anti-phosphotyrosine antibody (Santa Cruz; PY-20; sc-508) for phosphorylated FAK and c-Src. Densitometric analyses were performed using Adobe Photoshop (Adobe Systems Incorporated, San Jose, Calif.).

To assess the phosphorlyation status of ERK, 30 mg of whole cell lysates was loaded onto SDS-PAGE, and Western blot analysis was done using anti-p-ERK (Santa Cruz; E-4; sc-7383) and anti-ERK (Santa Cruz; K-23; sc-94) antibodies. To detect active RhoA, Rac1 and Cdc42, generated GST-tagged RhoA binding domain of Rhotekin protein (GST-Rhotekin-RBD) and GST-tagged p21-binding domain of p21-activated kinase 1 (PAK1)(GST-PAK1-PBD) were first generated. Briefly, E. coli BL21 cells transformed with pGEX4T-PAK1-PBD or pGEX4T-Rh,otekin-RBD were grown at 37° C. and expression of recombinant protein was induced by addition of 0.1 mM IPTG for 3 hours. Cells were resuspended in lysis buffer (50 mM Tris-HCl, pH 8.0, 10% glycerol, 20% sucrose, 2 mM DTT, 1 mg/ml leupeptin, 1 mg/ml pepstatin, and 1 mg/ml aprotinin), sonicated, and centrifuged at 4° C. for 30 min at 45,000 g. The supernatant was incubated with glutathione sepharose 4B beads (GE-Amersham, Piscataway, N.J.) for 1 hour at 4° C., and then washed 3 times in lysis buffer. Using these GST-proteins, GTPase activation assays were performed as described [6].

Briefly, cells resuspended in lysis buffer (50 mM Tris, pH8.0, 500 mM NaCl, 1% Triton X-100, 0.1% SDS, 0.5% sodium deoxycholate, 10% glycerol, 10 mM MgCl2, 10 mg/ml leupeptin and aprotinin, and 1 mM phenylmethylsulfornyl fluride) were cleared; the supernatants containing approximately 500 mg of protein were incubated with 5 mg of recombinant GST-Rhotekin-RBD- or GST8 PAK1-PBD (both conjugated to agarose beads) for 1 hr at 4° C., washed with lysis buffer, and eluted into SDS-loading buffer. Eluents, along with cleared cell lysates, were size fractionated by SDS-PAGE, transferred to nitrocellulose membranes, and probed by anti-RhoA, anti-Rac1, and anti-Cdc42 antibodies. Immunoreactivities on the eluents represent active forms of GTPases, whereas those on the total cell lysates represent total GTPases. These experiments were performed three times with the same results.

### Example 11

### Mouse Carotid Artery Injury Assay

The carotid artery injury assay was performed as described by Kuhel (16). Male apoE−/− mice were obtained from Jackson Laboratory (Bar Harbor, Me.) and maintained on a 12 hour light/dark cycle. All animal experimentation protocols were performed under institutional guidelines of animal welfare, in accordance with NIH guidelines. Mice were fed either normal rodent chow diet (5001, LabDiet) (n=10) or normal chow diet containing 0.15% morelloflavone (w/w) (n=9). This diet corresponded to 200 mg/kg morelloflavone if a 30 gm mouse consumes 4 grams of chow. Mice were placed on these diets for 7 days before they underwent carotid artery denudation by the insertion of an epoxy resin (Epon) probe as described (16).

Briefly, the entire length of the left carotid artery was exposed and the distal bifurcation of the carotid artery was looped proximally and ligated distally with a 7-0 suture. A transverse arteriotomy was made between the 7-0 silk sutures and the resin probe was inserted, advanced toward the aortic arch, and withdrawn 5 times. The probe was then removed, the proximal 7-0 suture was ligated, a 6-0 suture was secured, and the incision was closed with 5-0 sterile surgical gut. After surgery, mice were maintained on these diets 9 for 14 days before they were euthanized. After blood was sampled, the arteries of these mice were perfusion-fixed with 10% buffered formalin (pH 7.0) solution at a constant pressure of 100 mm Hg. The entire neck from each mouse was dissected, fixed further in 10% buffered formalin, decalcified, and then embedded in paraffin. Identical whole neck cross sections of 5 mm were made from the distal side of the neck beginning at the point of the distal 7-0 ligature. Whole neck sections were used to evaluate both the injured and the uninjured control vessels on the same section. For each mouse, the 4 levels of serial sections were taken at 500-mm intervals. These sections were stained by Verhoeff-van Gieson (VVG) staining and subjected to morphometric analyses. Images were digitized and captured using a Sony video connected to a personal computer.

Measurements were performed at a magnification of 200 using a Scion Image analysis computer program (Frederick, Md.). Data were obtained from the first 2 levels where endothelial denudation occurred. For each artery, the luminal area, area inside the internal elastic lamina (IELA), and the area encircled by external elastic lamina-IELA were measured. The intimal area (IA) was calculated as the IELA minus luminal area. For the determination of serum morelloflavone levels, animal sera were first digested by proteinase K in the presence of 0.01% SDS. Morelloflavone was then extracted into ethyl acetate and lyophilized.

The quantification of morelloflavone was performed by HPLC as described with following modifications. The pure morelloflavone was diluted to varying concentrations (0-100 mM), aliquoted into multiple tubes, and lyophilized. The samples were dissolved in 200 mL of 25% acetonitrile/0.55% acetic acid 10 of which 100 mL were injected into a Vydac C-18 reversed phase HPLC column (Grace Davison Discovery Sciences, Deerfield, Ill.) in an Agilent 1100 HPLC system (San Jose, Calif.). Morelloflavone was eluted from the column by a 25-55% acetonitrile/0.55% linear gradient with a flow rate of 1 ml/min with UV monitoring at 289 nm. The peaks were integrated and the signal to noise value was obtained from the HPLC software, using the baseline appearing after the morelloflavone peak to calculate noise. The limit of detection (LOD) is defined as the concentration of morelloflavone that yielded a signal to noise of 2:1, and was calculated at 1.06 mM, from the 20 mM standard peak (signal to noise=37.6:1). None of samples from control animals contained morelloflavone concentrations higher than the limit of detection.

For the analyses of intimal cell proliferation, apopotosis and ERK activation, Ki-67, Terminal deoxynucleotidyl transferase (TdT)-deoxyuridine nick-end labeling (TUNEL), and p-ERK staining, respectively, were performed. Ki-67 was detected using a monoclonal rabbit antibody (Clone TEC-3, DAKO North America, Inc., Carpinteria, Calif.) as described (8-9). Terminal deoxynucleotidyl transferase (TdT)-deoxyuridine nick-end labeling (TUNEL) staining (10) was performed using a FragEL™ DNA fragmentation detection kit (Oncogene Research Products, Boston, Mass.) according to the manufacturer's instructions. The Ki-67 and apoptotic indices, defined as the number of cells with DAB-positive nuclei divided by the total number of cells counted and expressed as a percentage, were then calculated. All cells within the intima were counted. Values are expressed as means±SD. Comparisons of parameters between two groups were made with Student's t test. When appropriate, ANOVA was performed to compare multiple groups. A value of P<0.05 was considered statistically significant.

### Example 12

### Animals

As a model of in vivo athereosclerosis, mice that lack the low density lipoprotein (LDL) receptor and Apobec 1 genes (Ldlr^{-/-}Apobec1^{-/-}) were used. The Ldlr^{-/-}Apobec1^{-/-} mice were generated by crossbreeding Ldlr^{-/-} mice (Jackson Lab, Bar Harbor, Me.) and Apobec1^{-/-} mice (Dr. Lawrence Chan, Baylor College of Medicine). Genotyping was performed in standard PCR-based methods, using following primer sets: For Apobec1, 5′-TGA GTG AGT GGT GGT GGT AAA G-3′ (SEQ ID NO: 1) and 5′-CGA AAT TCC TCC AGC AGT AAC-3′ (SEQ ID NO: 2) where Apobec1^{+/-} and Apobec1^{+/-} mice would have 475 by amplicon while Apobec1^{-/-} have none. For Ldlr, 5′-ACC CCA AGA CGT GCT CCC AGG ATG A-3′ (SEQ ID NO: 3), 5′-CGC AGT GCT CCT CAT CTG ACT TGT-3′ (SEQ ID NO: 4), 5′-AGG ATC TCG TCG TGA CCC ATG GCG A-3′ (SEQ ID NO: 5), and 5′-GAG CGG CGA TAC CGT AAA GCA CGA GG-3′ (SEQ ID NO: 6) where Ldlr^{+/+} mice would yield 383 bp amplicon, while Ldlr^{-/-} 200 bp and Ldlr^{+/+} 383 and 200 bp amplicons, respectively.

Mice lacking LDL receptor gene alone exhibit only mildly elevated LDL cholesterol level while Ldlr^{-/-}Apobec1^{-/-} mice exhibit drastically elevated LDL cholesterol levels and extensive atherosclerosis mimicking that of human on a standard chow diet. Twenty four (24) 8 week old Ldlr^{-/-}Apobec1^{-/-} mice were randomly assigned to either control group (normal rodent chow, Lab Diet, Richmond, Ind., N=12) or morelloflavone group (normal rodent chow supplemented with 0.003% (w/w) morelloflavone, N=12). The 0.003% (w/w) morelloflavone corresponds to approximately 4 mg/kg morelloflavone for 30 gram animals that consume 4 grams of chow. Animals were housed individually in an air-conditioned room with 12-h light/dark cycle with access to food and water. Body weight of these mice was monitored monthly. After 8 months, animals were sacrificed for analyses of atherosclerosis. Just prior to the sacrifice, blood was sampled from the heart and collected into micro-centrifuge tubes containing EDTA. The entire aortae were excised. All animal experimentation protocols were performed according to the protocol approved by the Institutional Animal Care and Use Committee (IACUC) of the University of Texas Health Science Center at Houston, in accordance with the National Institutes of Health guidelines.

### Example 13

### Analysis of Lipids

Plasma levels of triglycerides, total cholesterol, phospholipids, and non-esterified fatty acids of control and morelloflavone-treated animals were measured and using commercially available kits according to manufacturer's instruction (Randox Laboratories, Crumlin, UK; Thermo Fisher Scientific, Waltham, Mass.; Wako Chemicals, Richmond, Va.).

### Example 14

### Analysis of Atherosclerotic Lesions

The aortae and hearts were excised en bloc. The extent and degree of atherosclerosis was quantified by both en-face analyses of atherosclerosis lesions and the intimal areas of the ascending aortae at the level of aortic valve leaflet. For en-face analysis of atherosclerotic areas, the distal portion of the ascending aortae, aortic arches, and descending aortae down to the liac bifurcations were pinned flat on a white wax surface, fixed with 10% (v/v) buffered formalin solution overnight, stained by freshly prepared and filtered Oil red O solution for 1 hr, rinsed twice with 78% methanol, mounted and dried on the glass slides, and scanned in the TIFF format using the ScanScope slide scanning system (Nikon, Melville, N.Y.). The planimetry of the entire surface areas and Oil red O-positive atherosclerotic lesion areas was performed on the scanned images using Sigma Scan Pro software (SPSS Science, Chicago, Ill.).

For the cross sectional analyses of atherosclerosis, the roots of the ascending aortae were embedded in OCT compound (Tissue-Tek®, Sakura-Fineteck U.S.A., Torrance, Calif.). The 5-μm cryostat sections were obtained at the levels of aortic valve leaflets and subjected to hematoxylin and eosin (H&E), TUNEL, and other immunohistochemical staining as described below. In both methods of atherosclerosis quantification, the images of aortae were digitally captured and stored in the TIFF format, using the ScanScope slide scanning system (Nikon, Melville, N.Y.). The quantification of the atherosclerotic area was performed by Image J software (NIH, Bethesda, Md.) and expressed at lesion areas (μm²).

### Example 15

### Immunohistochemical Analysis of Atherosclerotic Lesions

In order to identify within the intima vascular smooth muscle cells, macrophages, and proliferating cells, the tissue was stained using anti-smooth muscle cell alpha-actin (SMA) (catalog no. ab5694, Abcam, Cambridge, Mass.), anti-macrophage surface glycoproteins (F4/80, catalog no. ab6640, Abcam), and anti-Ki67 (Clone TEC-3, DAKO, Carpinteria, Calif.) antibodies, respectively, as described previously. Values are expressed as means±SD. Comparisons of parameters between two groups were made with the two-tailed Student's t test. When appropriate, ANOVA was performed to compare multiple groups. A value of P<0.05 was considered statistically significant.

### Example 16

### Measurment of Thiobarbituric Acid Reactive Substance (TBARS)

EDTA-added mouse plasma was stored at −80° C. until the assay. The assay was performed using a commercially available kit according to the manufacturer's instructions. The degree of plasma lipid peroxidation was expressed in terms of equivalent malondialdehyde (MDA) concentration (Cayman Chemical Co., Ann Arbor, Mich.).

### Example 17

### Secreted Phospholipase A2 (sPLA₂) Enzyme Activity Assay

Recombinant human sPLA₂ isoforms, Group IB, Group IIA, Group V, and Lp-PLA₂ were obtained from R&D Systems and Cayman Chemical Co. sPLA₂ enzyme activities in the presence of various concentrations of morelloflavone or other inhibitors were determined using a commercially available assay system according to the manufacturer's instructions (Cayman Chemical Co.). Thioetheramide-1-palmitylthio-2-palmitoylamido-1,2-dideoxy-sn-glycero-3-phosphorylcholine (T-PC) and methyl arachidonyl fluorophosphonate (MAFP) were used as inhibitors of sPLA₂ (IB, IIA, and V) and Lp-PLA₂, respectively, per manufacturers' instructions.

### Example 18

### Statistical Analsysis

Values are expressed as means±standard deviation (SD). Comparisons of parameters between two groups were made with the two-tailed Student's t test. When appropriate, ANOVA was performed to compare multiple groups. A value of P<0.05 was considered statistically significant.

### Example 19

### Effect of Morelloflavone on Cell Cycle Progression

In order to investigate the biological effects of morelloflavone, high-purity morelloflavone was first prepared (94.3% by HPLC analyses, FIGS. 1B-1C), from the leaves of G. dulcis (L, FIG. 1A). To examine the effect of morelloflavone on cell cycle progression, vascular smooth muscle cells were treated with 0-100 mM morelloflavone and subjected them to flow cytometric analysis. Morelloflavone at concentrations up to 10 mM did not significantly affect progression of the cell cycle; however, at 100 mM, morelloflavone blocked G2 [symbol] M=G1 progression (FIG. 1D). BrdU incorporation assays consistently showed that morelloflavone did not significantly affect DNA synthesis at concentrations up to 10 mM (P=0.079; One-way ANOVA, Tukey's pairwise comparison) (FIG. 1E), together suggesting that morelloflavone, except at high concentrations, does not affect cell cycle progression or DNA synthesis of vascular smooth muscle cells.

To test the effect of morelloflavone on vascular smooth muscle cell viability, a MTT assay was performed. Morelloflavone showed no cytotoxicity at concentrations between 0 and 10 mM (P=0.071; One-way ANOVA, FIG. 1F). A standard DNA fragmentation-apoptosis assay showed that morelloflavone did not induce DNA fragmentation in vascular smooth muscle cells (FIG. 1G). Rather, DNA fragmentation indices decreased as morelloflavone concentrations increased (P=0.032, FIG. 1G).

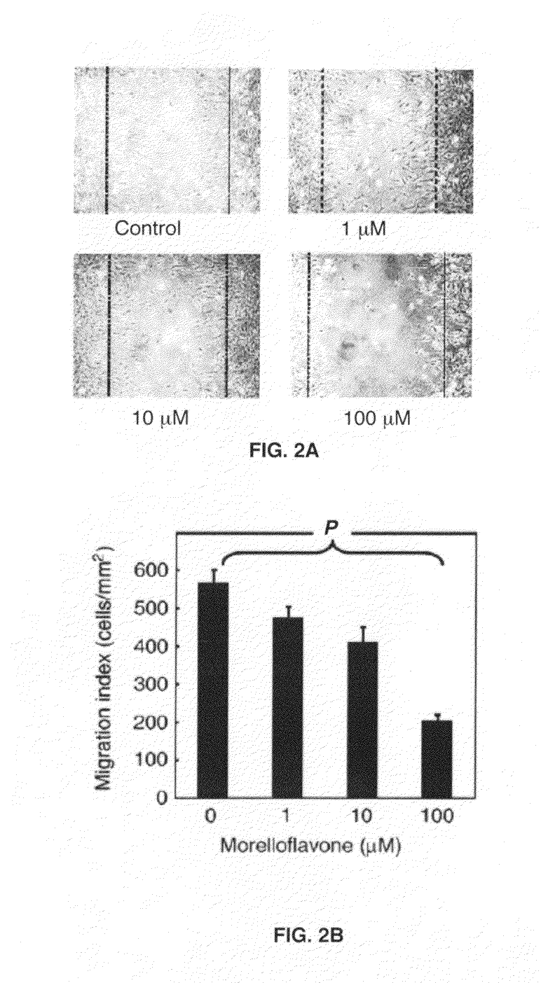

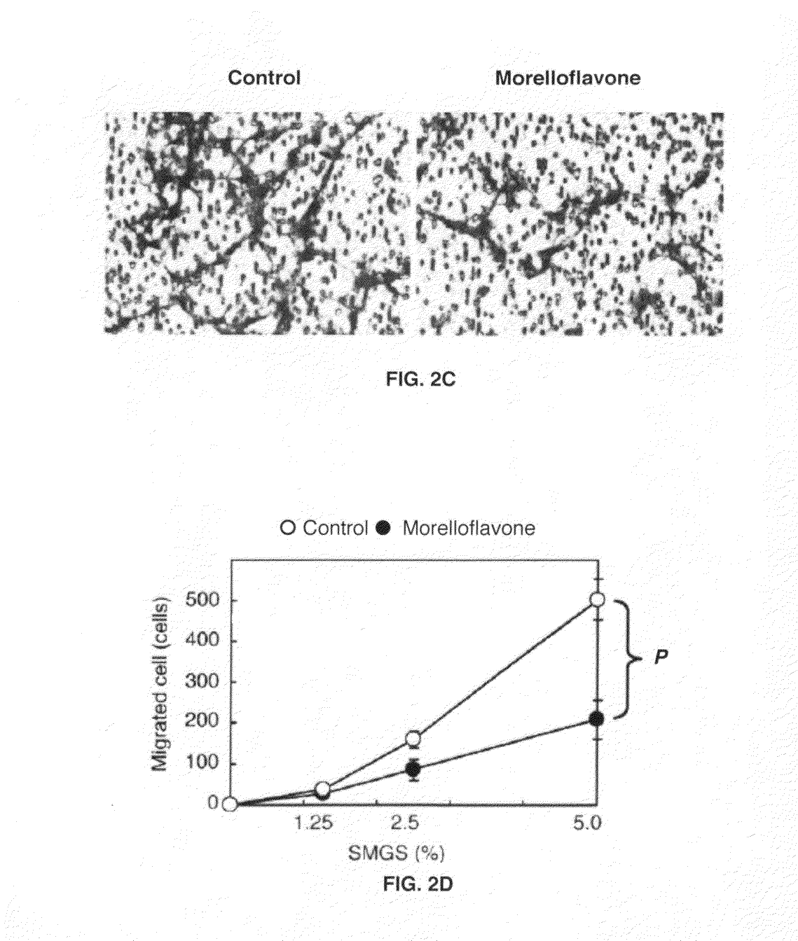

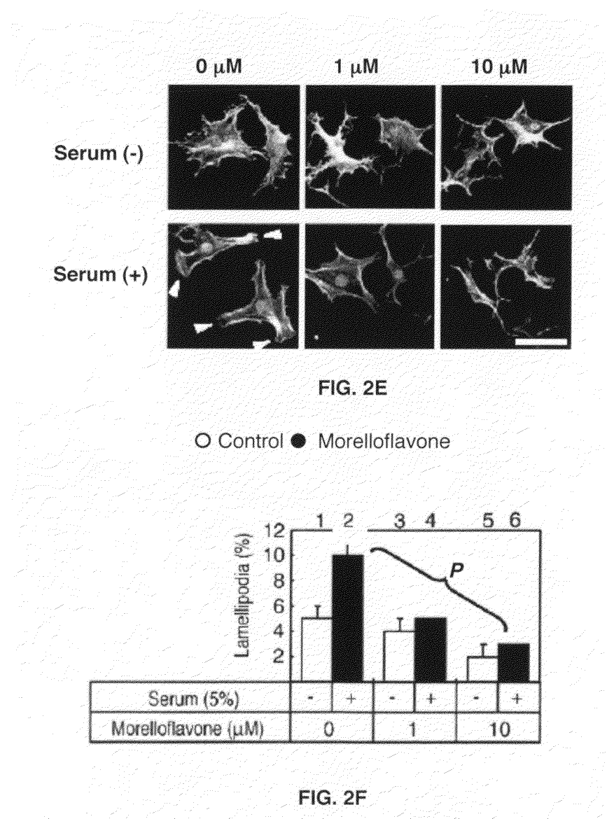

To determine whether morelloflavone plays a role in the regulation of vascular smooth muscle cell migration, a scratch wound assay was performed. As FIGS. 2A-2B shows, the migration of vascular smooth muscle cells was inhibited in a dose-dependent fashion (migration indices at 0, 1, 10 and 100 mM morelloflavone=568.7±33.5, 487.6±36.9, 411.3±39.5, and 191.86±32.8 cells/mm², respectively; P<0.001 by one-way ANOVA). In other words, there was a statistically-significant, negative correlation between morelloflavone concentrations and migration indices. In addition, there was a significant reduction in migration indices between 0 and 1 mM of morelloflavone (P<0.05, Tukey's pairwise comparison).

To evaluate the effect of morelloflavone on vascular smooth muscle cell invasion, a modified Boyden chamber assay was performed. While 0% of vascular smooth muscle cells did not cause vascular smooth muscle cells to migrate in the presence or absence of morelloflavone, the increasing concentrations of SMGS were associated with more migrated cells (FIGS. 2C-2D). In this system, morelloflavone at 1 mM vascular smooth muscle cell significantly blocked vascular smooth muscle cell migration (P=0.002 by two-way ANOVA). These findings, together with those presented in FIGS. 2A-2B, suggest that morelloflavone is a potent inhibitor of vascular smooth muscle cell migration and invasion.

The next step was to determine the effect of morelloflavone on the formation of the vascular smooth muscle cell migratory apparatus, or the lamellipodia (14-17). In the absence of serum, morelloflavone at any concentration did not change the morphology of vascular smooth muscle cells (FIG. 2E, Serum [−] at 0, 1, and 10 mM; FIG. 2F, columns 1, 3 and 5). In the absence of morelloflavone, the number of lamellipodia significantly increased upon serum stimulation (FIG. 2E, Serum [−] to [+] at morelloflavone 0 mM, FIG. 2F, columns 1 and 2; serum [−] vs. [+]=0.105±0.006 vs. 0.05±0.01; P<0.005). In this system, morelloflavone significantly decreased lamellipodium indices in a dose-dependent fashion (FIG. 2E, Serum [+] at 0, 1, and 10 mM; FIG. 2F, columns 2, 4, and 6=0, 1, and 10 mM morelloflavone=0.105±0.006, 0.05±0.001, 0.033±0.006; P<0.001 by one-way ANOVA).

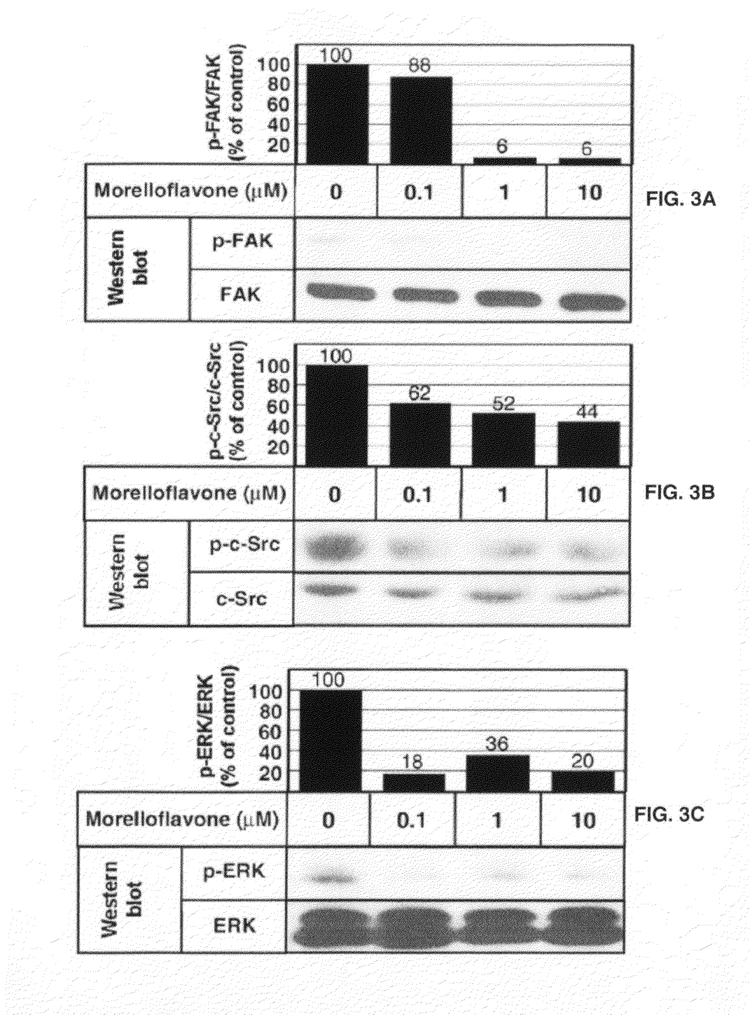

Next, the effect of morelloflavone on migration pathways, i.e., focal adhesion kinase (FAK) (18), c-Src (19), ERK (20), and small GTPases, RhoA, Rac1, and Cdc42 (21) was examined. Strikingly, morelloflavone inhibited the phosphorylation of focal adhesion kinase (FAK) and c-Src (FIG. 3A), the phosphorylation of ERK (FIG. 3B), and the activation of RhoA, all at low concentrations (0.1 & 1 mM) (FIG. 3C) was studied. Morelloflavone blocked the activation of Cdc42 at higher concentration (10 mM) and had no significant effects on Rac1 (FIG. 3C). In summary, morelloflavone blocks key migration-related kinases—explaining why morelloflavone can exert such a powerful inhibitory effect on migration as seen in FIGS. 2A-2F.

To assess whether morelloflavone's inhibitory effects on vascular smooth muscle cell migration in vitro could reduce injury-induced neointimal formation in a whole animal, apoE−/− mice were placed on normal chow (n=10) or chow containing morelloflavone (0.15% w/w, n=9) for 1 and 2 weeks, before and after endothelial denudation (16), respectively. ApoE−/− mice were used because they exhibit far more robust neointimal proliferation than do other mouse strains such as C57BL (22) and C3H (23) due to the fact that ApoE blocks injuryinduced neointimal proliferation via its suppression of cyclin D1 (23).

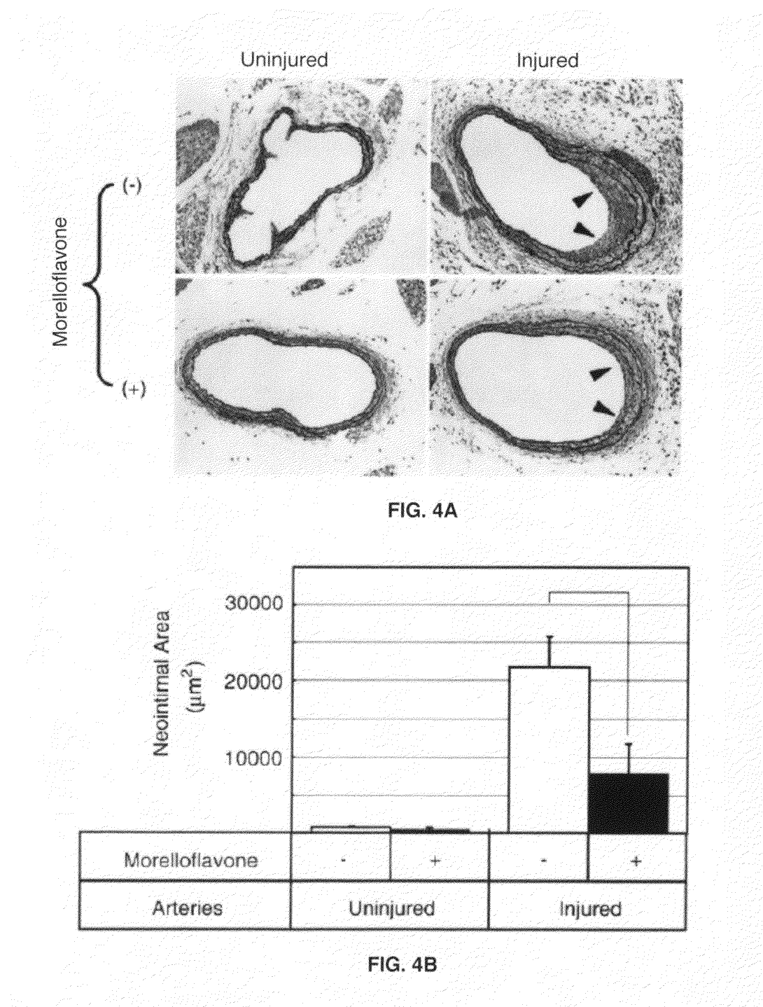

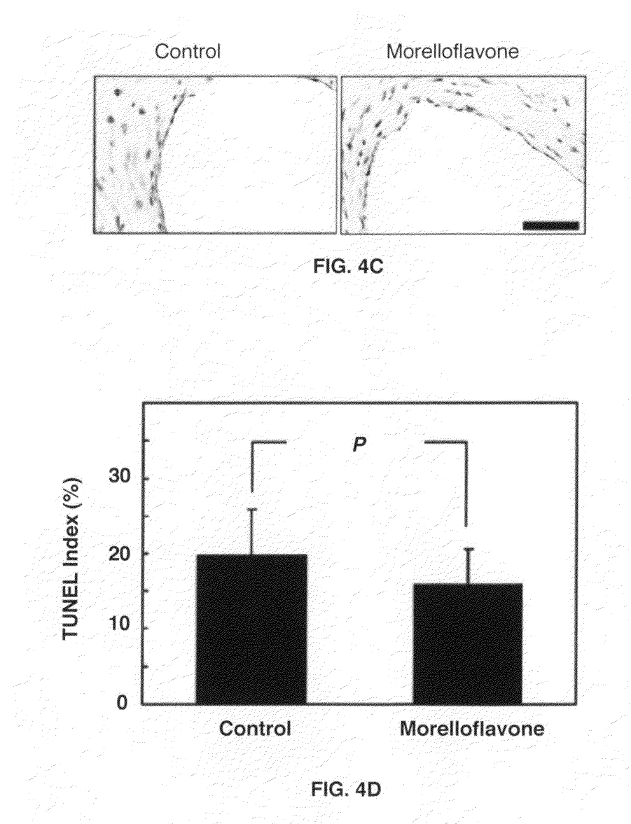

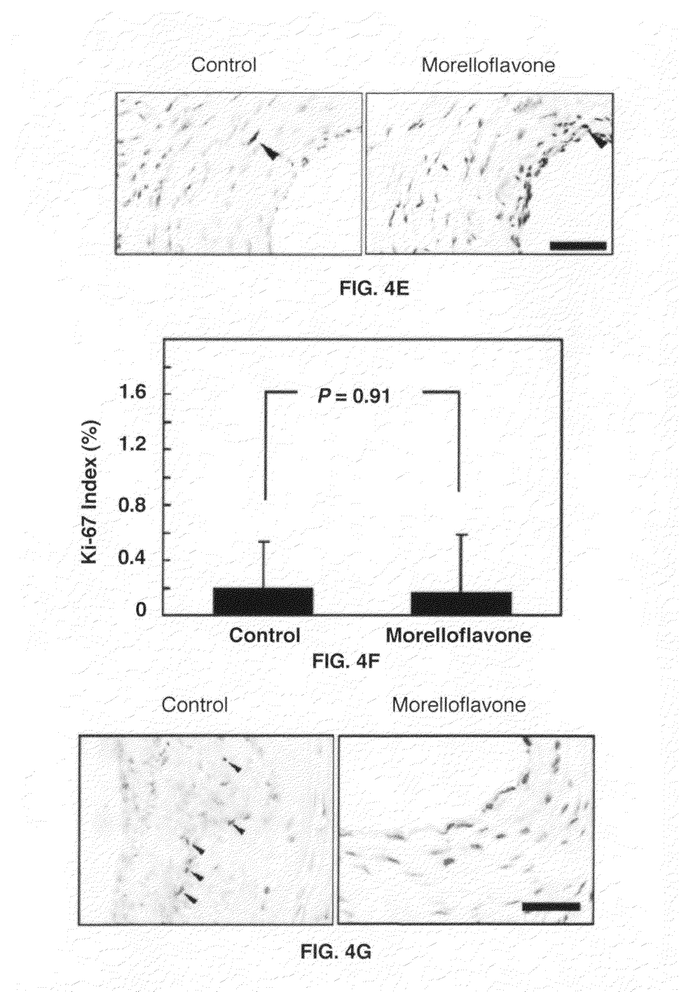

No significant differences in body weight were seen before injury (control vs. morelloflavone; 23.8±2.0 vs. 24.0±2.0 g, respectively, NS) or at the time of sacrifice (control vs. morelloflavone; 23.0±1.0 vs. 23.3±1.5 g, respectively, NS). The mean serum concentration of morelloflavone of treated animals was 1.37±0.78 mM. Morelloflavone treated mice had significantly less neointimal formation in injured carotid arteries than did control mice (Table 1 and FIGS. 4A-4B, control vs. morelloflavone; 21769.7±7862.7 mm2 vs. 7862.7±4047 mm2, respectively; P<0.01). TUNEL staining showed that there is no difference in TUNEL indices between control and morelloflavone groups (control vs. morelloflavone=19.9±6.1 vs. 16.0±4.6, P=0.23, FIGS. 4C-4D). Ki-67 staining failed to show any difference in Ki-67 indices between control and morelloflavone groups (control vs. morelloflavone=0.19±0.34 vs. 0.17±0.41%, P=0.91, FIGS. 4E-4F).

These data, combined with those presented in FIGS. 1A-1G and 2A-2F, suggest that morelloflavone reduced injury-induced neointimal formation by inhibiting vascular smooth muscle cell migration from the media to the intima in apoE−/− mice, but not by either increasing apoptosis or inhibiting cell proliferation in the neointima. In order to evaluate whether the inhibition by morelloflavone of ERK phosphorylation in vascular smooth muscle cells (FIG. 3B) can also be observed at a tissue level, immunostaining of p-ERK in these sections was performed. The p-ERK signals were seen in the neointima of 3 out of 7 tissue sections (42.9%) of control animals and in the neointima of one out of 8 sections (12.5%) of morelloflavone-treated animals (FIG. 4G), suggesting that oral morelloflavone decreases ERK activation in the neointima, a result concordant with what was observed in the tissue culture experiment (FIG. 3B). Morelloflavone or its derivatives, with further studies, may prove to be promising anti-restenotic agents. Centuries of its medicinal use in Thailand and others suggest that morelloflavone is well tolerated with minimal adverse effects.

TABLE 1
Morphometric Analyses of Uninjured and Injured Carotid Arteries

| Areas (mm²) | Treatment | Uninjured | Injured |
|---|---|---|---|
| Neointimal area | Control | 728.4 ± 796 | 21769.7 ± 11773 |
| | Morelloflavone | 512.0. ± 259 | 7862.7 ± 4047* |
| Medial area | Control | 26492.0 ± 8569 | 43902.7 ± 16916 |
| | Morelloflavone | 30230.6 ± 14393 | 39274.4 ± 15136 |
| Luminal area | Control | 60970.9 ± 4988 | 52743.6 ± 6954 |
| | Morelloflavone | 62340.2 ± 5904 | 62134.5 ± 10740 |
| Internal elastic | Control | 61699.3 ± 5099 | 74513.3 ± 6320 |
| lamina area | | | |
| | Morelloflavone | 62852.4 ± 5900 | 69997.2 ± 11145 |
| External elastic | Control | 88191.3 6255 | 118415.7 ± 12601 |
| lamina area | | | |
| | Morelloflavone | 93083.0 ± 9144 | 102971.6 ± 14326 |

*P < 0.01. The neointimal area was calculated as the internal elastic lamina area minus luminal area and the medial area as the external elastic lamia area minus the internal elastic lamina area.

### Example 20

### Morelloflavone Significantly Reduced Atherosclerosis

Ldlr^{-/-}Apobec1^{-/-} mice are severely hypercholesterolemic and spontaneously develop severe atherosclerosis on normal chow diet that closely mimics human atherosclerosis. In order to test the hypothesis that morelloflavone reduces atherosclerosis, 12 male Ldlr^{-/-}Apobec1^{-/-} mice were fed normal chow diet containing 0.03% (w/w) morelloflavone. The control was the group of 12 male Ldlr^{-/-}Apobec1^{-/-} mice that were fed with normal chow diet. Animals were kept on these diets for 8 months. Morelloflavone produced no changes in body weight (body weights at 8-month time point; control vs. morelloflavone=27.8±1.0 vs. 28.4±2.3 gm, NS) (Table 2). In addition, morelloflavone produced no changes in serum triglycerides, total cholesterol, phospholipid, and non-esterified fatty acid concentrations (Table 3). Both groups of mice had markedly increased levels of serum cholesterol (control vs. morelloflavone=512.6±102.0 vs. 507.2±95.8; normal range in male C57BU6 mouse=59±15 mg/dL (28)) (Table 3). The Ldlr^{-/-}Apobec1^{-/-} mice also had elevated serum triglycerides (control vs. morelloflavone=158.5±32.9 vs. 179.9±28.0; P=0.10; normal=56±12 (28)) and non-esterified fatty acid levels (control vs. morelloflavone=0.81±0.08 vs. 0.89±0.13; P=0.10; normal=0.39±0.1(28)).

TABLE 2
Body Weights in Animals

| | Control | Morelloflavone | |
|---|---|---|---|
| Time | (N = 12) | (N = 12) | P-value |
| Initial (0 month) | 22.7 ± 1.9 | 22.3 ± 1.4 | 0.60 |
| 1 month | 21.1 ± 1.9 | 22.6 ± 1.5 | 0.04 |
| 2 months | 23.4 ± 1.2 | 24.0 ± 1.6 | 0.28 |
| 3 months | 24.6 ± 1.2 | 25.4 ± 1.5 | 0.19 |
| 4 months | 25.3 ± 2.1 | 26.2 ± 1.5 | 0.24 |
| 5 months | 25.8 ± 1.7 | 26.8 ± 1.3 | 0.12 |
| 6 months | 26.8 ± 1.0 | 27.5 ± 1.7 | 0.24 |
| 7 months | 26.7 ± 1.4 | 28.2 ± 2.5 | 0.08 |
| Final (8 months) | 27.8 ± 1.0 | 28.4 ± 2.3 | 0.43 |

Values are expressed as mean ± SD.

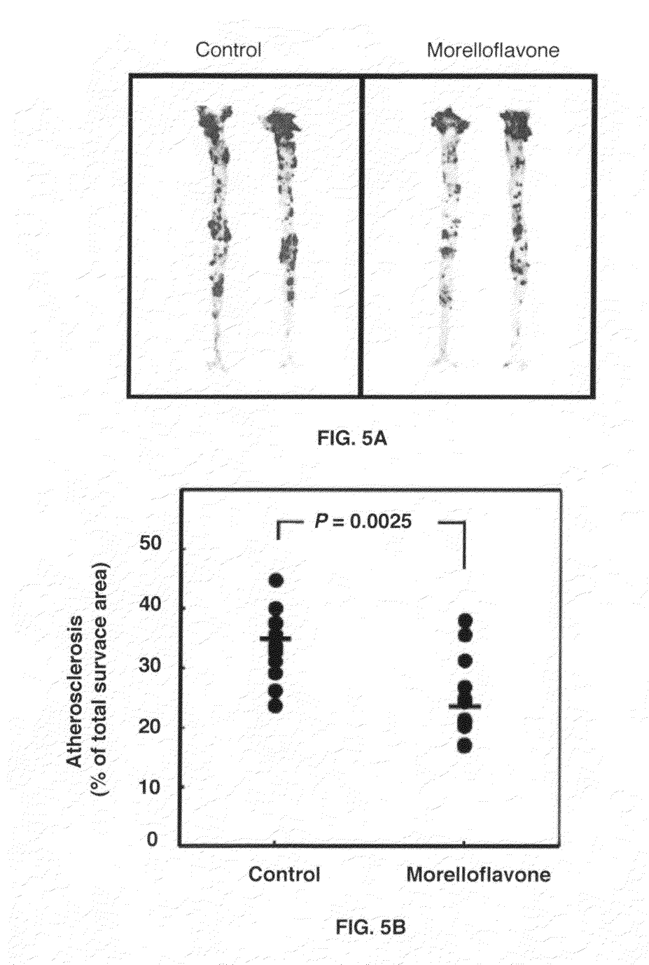

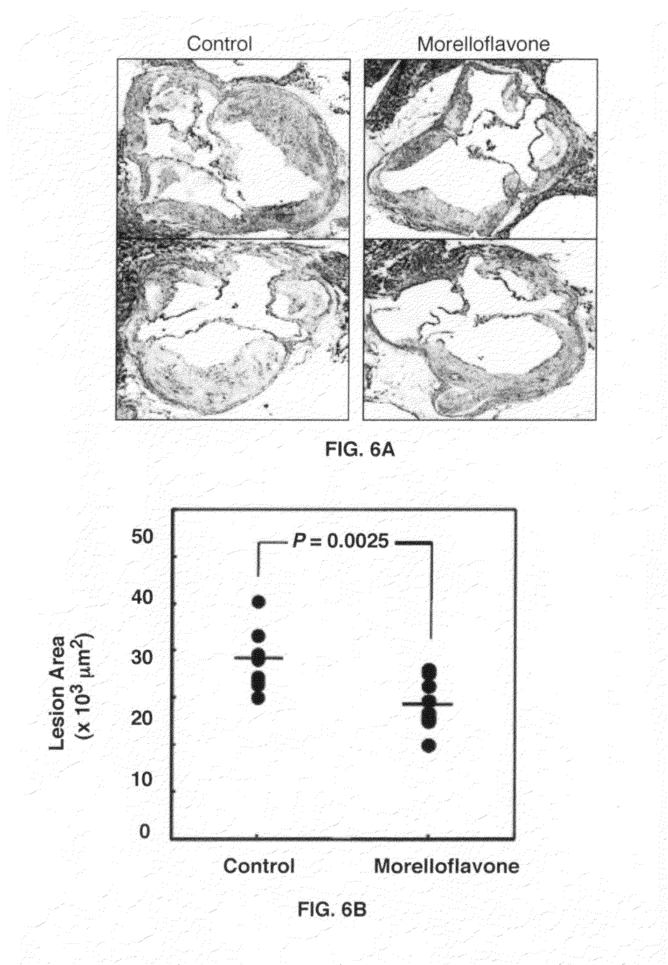

Atherosclerotic lesion size was measured in both the aortic intima by the en-face procedure as well as in sections of the aortic root at the level of aortic valve leaflets. Reductions in atherosclerosis were significant in both methods. By the en-face analysis, animals treated with morelloflavone had significantly lower atherosclerotic surface areas than control animals (control vs. morelloflavone=33.8±5.9 vs. 24.9±6.9 [% of the total aortic surface area], P=0.0025)—a 26% reduction (FIGS. 5A-5B). By the cross-sectional lesion area analyses, animals treated with morelloflavone had significantly smaller atherosclerotic surface areas than control animals (control vs. morelloflavone=7.64±1.30 vs. 5.65±1.05 [×10³ μm²], P=0.0025)—a 26% reduction (FIGS. 6A-6B). These data, taken together, suggest that oral, long-term morelloflavone treatment is associated with significantly reduced atherosclerosis in male, chow-fed Ldlr^{-/-}Apobec1^{-/-} mice.

### Example 21

Immunostaining of Atherosclerotic Tissue in the Aortic Roots Showed Significantly Less Vascular Smooth Muscle Cells in the Lesions of Morelloflavone-Treated Mice

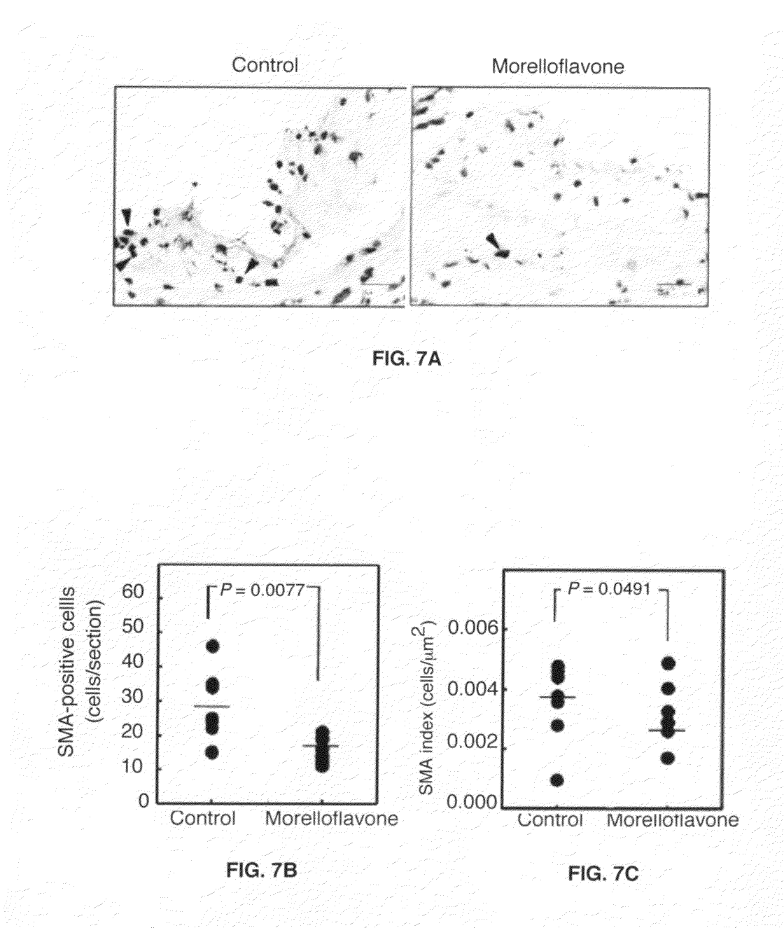

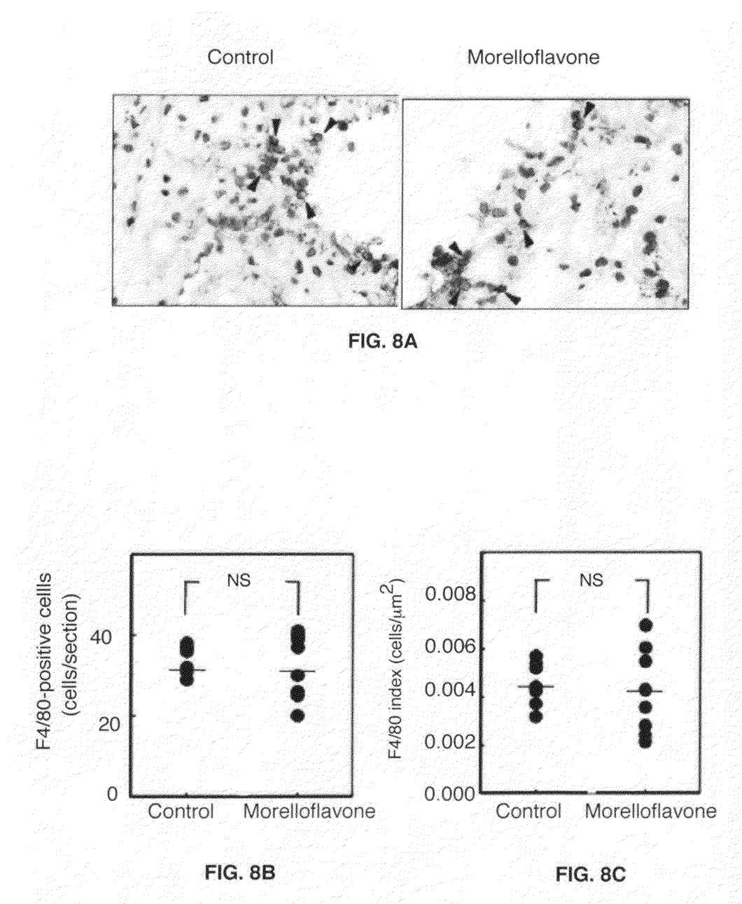

In order to determine whether reductions in atherosclerotic lesions by morelloflavone were accompanied by changes in cell composition, immunostaining of vascular smooth muscle cells and macrophages was performed. As shown in FIGS. 7A-7C, morelloflavone reduced the number of SMA-positive cells (VSMCs) in the atherosclerotic lesions (control vs. morelloflavone=27.0±10.7 vs. 15.4±3.5 [cells/section], P=0.0077, a 43% reduction; 0.0038±0.0014 vs. 0.0026±0.0007 [cells/μm²], P=0.0491, a 32% reduction), suggesting that morelloflavone treatment was associated with vascular smooth muscle cells infiltration in atherosclerotic plaques. However, morelloflavone did not change the number of macrophages in the lesions (control vs. morelloflavone=33.6±3.8 vs. 32.3±8.0 [cells/section], NS; 0.0045±0.0009 vs. 0.0042±0.0018 [cells/μm²], NS) (FIGS. 8A-8C), suggesting that morelloflavone treatment did not change macrophage infiltration in atherosclerotic plaques.

### Example 22

### Ki-67 and TUNEL Staining Showed that Morelloflavone Did not Affect Proliferation or Apoptosis of Cells within Atherosclerotic Tissue in the Aortic Roots

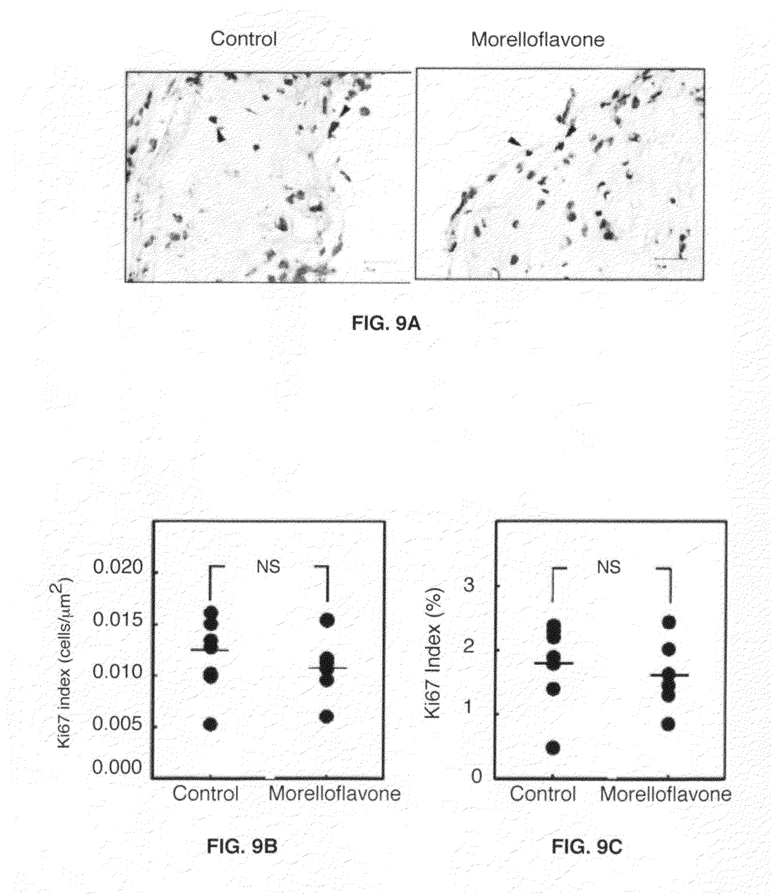

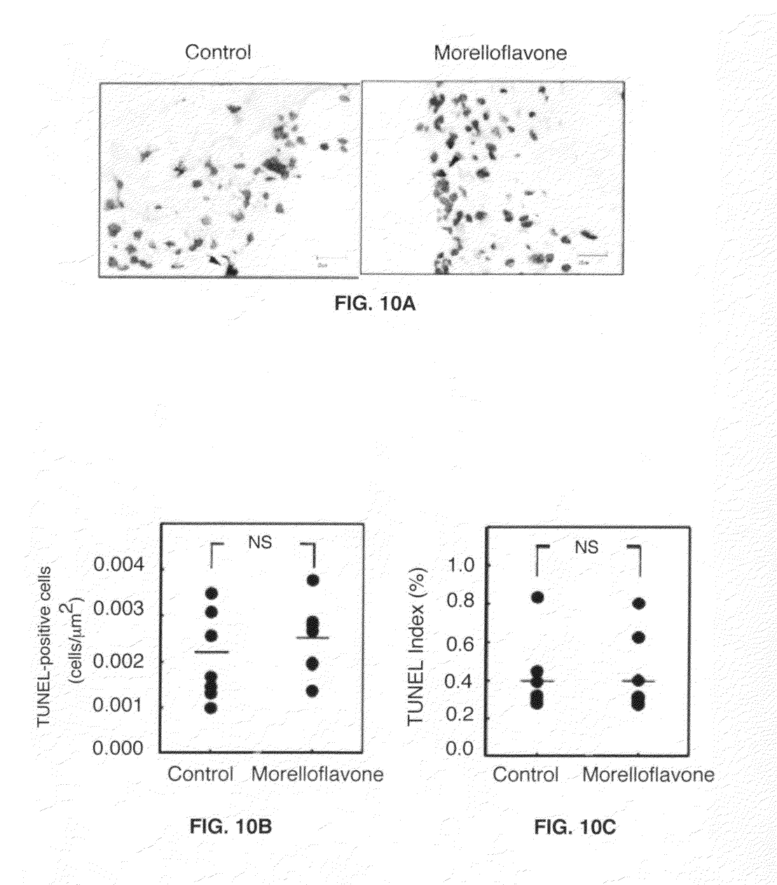

In order to evaluate whether morelloflavone affected proliferative and apoptotic responses of cells within the atherosclerotic lesions, aortic root sections were subjected to Ki-67 and TUNEL staining. As is shown in FIGS. 9A-9C and FIGS. 10A-10C, morelloflavone did not change the number of Ki-67-positive cells (control vs. morelloflavone=0.0013±0.0003 vs. 0.0011±0.0003 [cells/μm²], NS; 1.8±0.7 vs. 1.6±0.5 [Ki-67 index, %], NS) or TUNEL-positive (control vs. morelloflavone=0.00021±0.0001 vs. 0.00025±0.00008 [cells/μm²], NS; 0.4±0.2 vs. 0.4±0.2 [TUNEL index, %], NS) cells. These data suggest that morelloflavone treatment is not associated with changes in proliferative and apoptotic responses within atherosclerotic lesions.

### Example 23

### Morelloflavone does not Affect the Degree of Plasma Lipid Peroxidation

A standard thiobarbituric acid reactive substances (TBARS) assay was performed to assess the overall status of plasma lipid peroxidation in morelloflavone-treated and control mice. Thiobarbituric acid reactive substances concentration was 6.78±1.19 [μM] for control mice (N=12) while it was 6.33±1.47 [μM] for morelloflavone-treated mice (N=12, P=0.42). This suggests that morelloflavone, present in plasma at a sufficient concentration to reduce atherosclerosis, did not significantly reduce plasma lipid peroxidation.

### Example 24

### Morelloflavone does not Inhibit sPLA2-IIa In Vitro

The ability of morelloflavone to inhibit various phospholipase A2 (PLA₂), i.e., sPLA₂-IB, sPLA₂-IIA, sPLA₂-V, and Lp-PLA₂, was tested in vitro using recombinant human enzymes (data not shown). Morelloflavone minimally inhibited sPLA₂-IB, sPLA₂-IIA and sPLA₂-V, with IC₅₀ exceeding 10 μM. Morelloflavone did not inhibit Lp-PLA₂.

### Discussion

The effect of oral morelloflavone on atherogenesis was evaluated using Ldlr^{-/-}Apobec1^{-/-} mice and it was found that morelloflavone reduced atherosclerogensis in the model. While ApoE^{-/-} and Ldlr^{-/-} mice have been used as mouse models of atherosclerosis, Ldlr^{-/-}Apobec1^{-/-} mice were used in the current study. Ldlr^{-/-}Apobec1^{-/-} mice lack the apoB mRNA editing catalytic polypeptide-1 (apoBEC-1) and LDL receptors (LDLR). Deletion of LDLR in mice (Ldlr^{-/-} mice) leads to modest hypercholesterolemia and do not develop considerable atherosclerotic lesions when maintained on normal diets. This comes from the fact that the mouse liver produces lipoprotein containing a truncated form of apolipoprotein B48 due to the action of apoBEC-1. Unlike LDLr^{-/-} mice, these double genetically manipulated mice, deficient for both apoBEC-1 and LDLR, have markedly increased plasma cholesterol concentrations and develop extensive lesions throughout the aorta including most of the branch points that mirror pathophysiology of human familial hypercholesterolemia. The infiltration of vascular smooth muscle cells represents the late event of atheroscierogenesis. It is likely that morelloflavone, by inhibiting the infiltration of vascular smooth muscle cells into developing atherosclerotic lesions, ameliorated the progression of atherosclerosis in Ldlr^{-/-}Apobec1^{-/-} mice.

The biological effects of morelloflavone on vascular smooth muscle cells was characterized in vitro tissue culture and in vivo injured mouse arteries. Morelloflavone has unique biological properties; it does not affect cell cycle progression or cell survival at concentrations up to 10 μM, whereas it has profound effects on migration at a concentration as low as 1 μM. The potential mechanisms of morelloflavone's negative effect on migration include the de-activation of the migration-related molecules such as FAK, c-Src, ERk, and RhoA. Strikingly but consistently, oral administration of morelloflavone reduced neointimal formation in injured mouse carotid arteries without affecting the degree of apoptosis or proliferation of neointimal cells.

Although the extraporation of the current findings in a mouse model of atherosclerosis to the clinical usefulness must be done with caution, morelloflavone or its derivatives, with further studies, may prove to be promising oral anti-atherosclerotic agents. For example, morelloflavone could be administered orally as a secondary preventive measure for patients who, despite their normal cholesterol levels, suffer from coronary artery disease. Morelloflavone is well tolerated with an acceptable toxicology profile and minimal adverse effects and suited for long-term administration.

The following references were cited herein:
1. Morimoto, S. et al. (1990). Jpn Circ J 54, 43-56.
2. Nobuyoshi, et al., (1991). J Am Coll Cardiol 17, 433-9.
3. Karsch, et al., (1991). J Am Coll Cardiol 17, 991-4.
4. Subhadrabandhu, S. (2001) Under-utilized tropical fruits of Thailand (Nations, F.a.A.O.o.t.U., ed). RAP Publication
5. Deachathai, et al., (2006). Phytochemistry 67, 464-9.
6. Deachathai, et al., (2005). Phytochemistry 66, 2368-75.
7. Verbeek, et al., (2004). Biochem Pharmacol 68, 621-9.
8. Lin, Y. M. et al. (1997). J Nat Prod 60, 884-8.
9. Gil, et al., (1997). Biochem Pharmacol 53, 733-40.
10. Hutadilok-Towatana, et al., (2007). Nat Prod Res 21, 655-62.
11. Sanz, M. J. et al. (1994). Xenobiotica 24, 689-99.
12. Phongpaichit et al., (2006) FEMS Immunol Med Microbiol 48, 367-72.
13. Ross, R. (1971). J Cell Biol 50, 172-86.
14. Liu, et al., (2006). Circ Res 98, 480-9.
15. Guo, X. et al. (2003). J Biol Chem 278, 13207-15.
16. Kuhel, et al., (2002). Arterioscler Thromb Vasc Biol 22, 955-60.
17. Small, et al., (2002). Trends Cell Biol 12, 112-20.
18. Ilic, D. et al. (1995). Nature 377, 539-44.
19. Klinghoffer, et al., (1999). Embo J 18, 2459-71.
20. Nguyen, et al., (1999). J Cell Biol 146, 149-64.
21. Gerthoffer, W. T. (2007). Circ Res 100, 607-21.
22. Zhu, et al., (2002). Arterioscler Thromb Vasc Biol 22, 450-5.
23. Zhu, et al., (2000). Am J Pathol 157, 1839-48.

Any patents or publications mentioned in this specification are indicative of the levels of those skilled in the art to which the invention pertains. These patents and publications are herein incorporated by reference to the same extent as if each individual publication was individually incorporated by reference.

One skilled in the art will readily appreciate that the present invention is well adapted to carry out the objects and obtain the ends and advantages mentioned, as well as those inherent therein. It will be apparent to those skilled in the art that various modifications and variations can be made in practicing the present invention without departing from the spirit or scope of the invention. Changes therein and other uses will occur to those skilled in the art which are encompassed within the spirit of the invention as defined by the scope of the claims.

What is claimed is:

1. A method for treating postangiplasty or in-stent restenosis in an individual in need of such treatment, comprising the step of administering to said individual an effective dose of morelloflavone.

2. The method of claim 1, wherein said morelloflavone retards the progression of atherosclerosis.

3. The method of claim 1, wherein said morelloflavone retards migration of vascular smooth muscle cells.

4. The method of claim 1, wherein said morelloflavone decreases activation of ERK, RhoA, Rac, FAK and cSrc.

5. The method of claim 1, wherein said individual is at risk for percutaneous coronary intervention.

6. The method of claim 1, wherein said individual has atherosclerosis in coronary arteries, cerebral arteries, renal arteries, aorta, or peripheral arteries.

7. The method of claim 1, wherein said morelloflavone is administered orally.

8. The method of claim 1, wherein said morelloflavone is administered is a dose of from about 0.1 mg/kg to about 100 mg/kg of the individual's body weight.

9. The method of claim 1, further comprising the step of administering an HMG-CoA reductase inhibitor.

10. The method of claim 8, wherein said statin is selected from the group consisting of Atorvastatin, Cerivastatin, Fluvastatin, Lovastatin, Mevastatin, Pitavastatin, Pravastatin, Rosuvastatin, Simvastatin, Simvastatin.

11. A method for treating postangiplasty or in-stent restenosis in an individual in need of such treatment, comprising the step of administering to said individual an effective dose of morelloflavone in a dose of from about 0.1 mg/kg to about 100 mg/kg of the individual's body weight.

12. A method for treating postangiplasty or in-stent restenosis in an individual in need of such treatment, comprising the step of administering to said individual an effective dose of morelloflavone and an HMG-CoA reductase inhibitor.

13. The method of claim 12, further comprising administering an HMG CoA reductase inhibitor or a hypolipidemic agent or lipid-lowering agent or other lipid agent or lipid modulating agent or anti-atherosclerotic agent.

14. The method of claim 13, wherein said hypolipidemic agent or lipid-lowering agent or other lipid agent or lipid modulating agent or anti-atherosclerotic agent is selected from the group consisting of 1,2,3 or more MTP inhibitors, squalene synthetase inhibitors, fibric acid derivatives, PPAR .alpha. agonists, PPAR dual .alpha./.gamma. agonists, PPAR .delta. agonists, ACAT inhibitors, lipoxygenase inhibitors, cholesterol absorption inhibitors, ileal Na.sup.+/bile acid cotransporter inhibitors, upregulators of LDL receptor activity, cholesteryl ester transfer protein inhibitors, bile acid sequestrants, or nicotinic acid and derivatives thereof, ATP citrate lyase inhibitors, phytoestrogen compounds, an HDL upregulators, LDL catabolism promoters, antioxidants, PLA-2 inhibitors, antihomocysteine agents, HMG-CoA synthase inhibitors, lanosterol demethylase inhibitors, or sterol regulating element binding protein-I agents.

15. The method of claim 12, further comprising administering a platelet aggregation inhibitor.

16. The method of claim 15, wherein said platelet aggregation inhibitor is selected from the group consisting of aspirin, clopidogrel, ticlopidine, dipyridamole, ifetroban, abciximab, tirofiban, eptifibatide, anagrelide, CS-737, melagatran, ximelagatran, razaxaban.

17. A pharmaceutical composition comprising a morelloflavone and a pharmaceutically acceptable carrier therefor.

18. A pharmaceutical combination comprising a morelloflavone and one of:
a HMG CoA reductase inhibitor compound;
a hypolipidemic agent or lipid-lowering agent or other lipid agent or lipid modulating agent or anti-atherosclerotic agent; or
a platelet aggregation inhibitor.

19. A method for treating atherosclerosis in an individual in need of such treatment, comprising the step of administering to said individual an effective dose of morelloflavone.

20. The method of claim 19, wherein said morelloflavone retards migration of vascular smooth muscle cells.

21. The method of claim 19, wherein said individual has atherosclerosis in coronary arteries, cerebral arteries, renal arteries, aorta, or peripheral arteries.

22. The method of claim 19, wherein said morelloflavone is administered orally.

23. The method of claim 19, wherein said morelloflavone is administered is a dose of from about 0.1 mg/kg to about 100 mg/kg of the individual's body weight.

24. The method of claim 19, further comprising the step of administering an HMG-CoA reductase inhibitor.

25. The method of claim 24, wherein said statin is selected from the group consisting of Atorvastatin, Cerivastatin, Fluvastatin, Lovastatin, Mevastatin, Pitavastatin, Pravastatin, Rosuvastatin, Simvastatin, Simvastatin.
